(19) United States
(12) Patent Application Publication
(10) Pub. No.: US 2007/0258990 A1
(43) Pub. Date: Nov. 8, 2007

(54) Means and Methods for Detecting and/or Staging Follicular Lymphoma Cells

(76) Inventors: Daphne de Jong, Haarlem (NL); Annuska Maria Glas, Uitgeest (NL); Lodewyk Frederik Ary Wessels, Delft (NL)

Correspondence Address: TRASK BRITT, P.O. BOX 2550, SALT LAKE CITY, UT, 84110, US

(21) Appl. No.: 11/660,482

(22) PCT Filed: Aug. 18, 2005
(86) PCT No.: PCT/NL05/00602

§ 371 Date: Jul. 2, 2007

(30) Foreign Application Priority Data
Aug. 18, 2004 (EP) 04077343.4

(51) Int. Cl. C12Q 1/68; A61K 39/00

(52) U.S. Cl. 424/184.1; 435/6

(57) ABSTRACT

The invention provides a method for detecting and/or staging a malignant functional cell comprising measuring an expression profile of said malignant cell and a non-malignant cell. Preferably, said non malignant cell and said malignant cell are capable of interacting. In one aspect the invention provides reporter genes whose expression patterns are indicative for the presence and/or stage of follicular lymphoma.

The invention relates to the field of diagnostics. More specifically the invention relates to determination of the presence and/or stage of a malignant cell.

Malignancies are a major cause of mortality world wide. Much research has been undertaken to gain insight into origin, development and characteristics of many different kind of malignancies. Based on this insight diagnostic and therapeutic methods have been developed. However, a malignancy at various stages requires different kind of treatment.

In view of the different characteristics of a malignancy at various stages, a reliable test for determining the presence, kind and/or stage of a malignancy is of utmost importance. Once the kind and stage of a malignancy is known, an optimal therapy can be chosen which sufficiently counteracts the malignancy while avoiding over-treatment in order to minimize harmful side-effects. However, the stage of a malignancy is not always reliably determined. For instance, morphologically grading of a malignancy by a pathologist does not always have a good reproducibility due to the subjective nature of morphological grading. The cellular morphology of malignant cells is highly variable in a significant number of cases and the malignant cell component is often heterogeneous within a biopsy.

Recently, a new method for diagnosing the presence and/or stage of malignancies has been developed. Expression profiles of a number of genes of malignancies are compared with reference expression profiles. The differences are used to evaluate the presence and/or stage of disease (WO 02/103320). This kind of diagnosis does not provide results for all kinds of malignancies. For instance, this kind of test does not provide results for determining the presence and/or stage of follicular lymphoma. According to the present invention, an expression profile of malignant cells and surrounding non-malignant cells is measured.

It is an object of the present invention to provide a method for detecting a malignant cell. It is a particular object to provide a method for staging a malignant cell.

The invention provides a method for detecting and/or staging a malignant functional cell comprising measuring an expression profile of said malignant cell and a non-malignant cell.

The invention provides the insight that the presence and/or stage of a malignant functional cell is determined by measuring an expression profile of said malignant cell and a non-malignant cell. Hence, although prior art methods focus on determining characteristics that are only attributable to a malignant cell, and which often distinguish said malignant cell from other non-malignant cells, the present invention provides the insight that an expression profile of a malignant functional cell and a non-malignant cell provides insight into the presence and, preferably, the stage of said malignant cell.

A malignant functional cell is defined herein as a malignant cell which is not dedifferentiated to such extent that all original functions except replication are lost. Said functional cell is still capable of performing at least one similar function—in kind, not necessarily in amount—as compared to the same kind of cell that is not malignant. Preferably, a malignant functional cell of the invention is still capable of interacting with adjacent tissue. In one embodiment adjacent, surrounding cells are capable of influencing at least one characteristic of said malignant functional cell. Preferably said adjacent, surrounding cells are capable of influencing proliferation of said malignant functional cell. In a further embodiment said malignant functional cell is capable of influencing at least one characteristic of said non-malignant cell. In one embodiment said malignant functional cell is capable of influencing at least one characteristic of an adjacent, surrounding non-malignant cell and an adjacent, surrounding cell is capable of influencing at least one characteristic of said malignant functional cell. In this embodiment an expression profile of said malignant cell and said non-malignant cell is preferably determined. In one embodiment an expression profile of said adjacent non-malignant cells is determined.

One typical example of a malignant functional cell of the invention is a follicular lymphoma cell. The natural course of waxing and waning disease in the early phases of follicular lymphoma indicates that the proliferation of follicular lymphoma cells is not fully autonomous and that the process is relatively dependent on exogenic factors, e.g. immunological drive. Clinical behavior in follicular lymphoma (FL) is partly determined by factors related to the malignant cells (genetic factors) and partly by non-malignant cells such as surrounding T-cells and immunological accessory cells. Follicular lymphoma cells are, at least in early stages, still capable of interacting with surrounding cells and they are still immunologically active, at least to some extent. Therefore, FL is a disease of immunologically functional cells. The growth properties are at least partially determined by immunological support.

Another typical example of a malignant functional cell of the present invention is a gastric marginal zone lymphoma of MALT-type (MALT-lymphoma). Gastric MALT-lymphoma derives from precursor populations in follicular Helicobacter pylori-associated gastritis, that through accumulating genetic changes ultimately develop into an autonomously proliferating, monoclonal B-cell lymphoma. In the early phases of the disease, the proliferation is still at least partly dependent on the presence of Helicobacter pylori-induced T-cell help as is supported by in vitro evidence as well as by in vivo evidence of clinical malignancy regression after eradication of H. pylori. Hence, at these stages a gastric MALT-lymphoma is not fully dedifferentiated and fully autonomous, but requires interaction with surrounding T cells for proliferation; such surrounding T cells are a good example of a non-malignant cell of the present invention.

Certain types of solid malignancies have retained at least part of their functional immunological properties and are therefore typical examples of a malignant functional cell of the invention. Renal cell carcinoma (Grawitz tumor) and melanoma often have functional antigen-presenting properties. Another preferred example of a malignant functional cell of the invention is a prostate cancer cell.

Staging of a malignant functional cell of the invention, also called grading of a malignant functional cell of the invention, means classification of a certain malignancy. The stage of a malignancy is determined. For instance, in case of follicular lymphoma, it is determined whether the malignancy is graded as 1, 2, 3a or 3b. With a method of the invention it has become possible to accurately stage a malignant functional cell. Especially when a follicular lymphoma has grade 2 or 3, a method of the invention is more accurate as compared to morphological grading. It has become possible to more reliably distinguish between FL that behave clinically indolent and those that behave aggressively. Hence, a method of the invention is also suitable when a biopsy with inconclusive morphological features is obtained, from which a pathologist cannot derive meaningful information for morphological grading. Once the stage is known, adapted therapy can be provided. Hence, a method of the invention allows for reliable diagnosis even in cases where morphological grading is ambiguous.

With a method of the invention an expression profile is measured. This means that it is determined whether at least one reporter gene is expressed by said malignant functional cell and/or said non-malignant cell. Preferably, expression of a plurality of reporter genes is examined. In one embodiment the invention provides a method of the invention comprising determining the presence and/or amount of an expression product of at least two reporter genes in a sample comprising said malignant functional cell and at least one non-malignant cell. Said malignant functional cell and said non-malignant cell are preferably sampled together. Preferably, a biopsy containing both said malignant cell and surrounding non-malignant cells is taken.

In one embodiment, a malignant functional cell is detected and/or staged by detecting a difference in expression of at least one reporter gene as compared to a reference and/or control. Said reference and/or control for instance comprises an expression profile of the same kind of functional cells that are not malignant and the same kind of non-malignant cells (herein called “healthy reference and/or control”). Alternatively, said reference and/or control comprises an expression profile of the same kind of malignant cells which have a specific, known, stage and in the same kind of non-malignant cells (herein called “stage reference and/or control”). An expression profile of a sample comprising said malignant cells and said non-malignant cells is compared to at least one reference and/or control. If an expression profile of a sample is significantly different from a healthy reference and/or control, it is indicative for the presence of malignant functional cells and/or the stage of said malignant functional cells. If an expression profile of a sample is essentially the same as a certain stage reference and/or control, it is indicative for the presence of malignant functional cells of the same stage. An expression profile of a sample is “essentially the same” if the amount of expression product of at least one gene of said sample involved in a certain pathway is comparable to the amount of expression product of at least one gene of said reference and/or control involved in the same kind of pathway. Preferably, the amount of expression product of at least one specific gene of said sample is comparable to the amount of expression product of the same kind of gene of said reference and/or control. Most preferably, the amount of expression product of a plurality of genes of said sample is comparable to the amount of expression product of the same kind of genes of said reference and/or control. In one embodiment, the amount of expression product present of a sample is quantified.

In a preferred embodiment an expression profile of at least one reporter gene which is involved with a proliferation/apoptosis pathway, a metabolism pathway, an immune competence pathway and/or a T cell infiltration pathway is measured, since genes involved with these pathways have a strong prognostic/diagnostic value in a method according to the invention.

In one embodiment said expression product comprises RNA, preferably mRNA. The amount of mRNA is particularly indicative for the extent of expression of a reporter gene. Many methods are known in the art for determining the presence of RNA. For instance, RNA is isolated from a sample, after which an amplification reaction such as Nasba or RT-PCR is performed. RNA is for instance detected and/or quantified using internal calibrators, Taqman probes and/or beacon probes. To achieve quantification, a dilution series of target sequence is for instance amplified and the time points at which amplified nucleic acid becomes measurable (the time to positivity, TTP) are plotted against the input amounts of nucleic acid. This way a calibration curve is created that is suitable for correlating TTP values of reactions with unknown amounts of input with input amount. Many alternative methods for detecting and/or quantifying RNA present in a sample are available in the art. Preferably, protocols for RNA isolation, amplification, labeling and hybridization as described at http://www.nki.nl/nkidep/pa/microarray are used. These protocols are incorporated in the examples. RNA may be analyzed directly or via an intermediate such as a DNA copy, such as cDNA or a protein copy. A protein copy can be generated from said RNA directly or via the generation of a DNA copy of the RNA. In case intermediates are analyzed, either a sense or an anti-sense copy, or both can be detected.

In another embodiment said expression product comprises protein. An expression profile is for instance generated using western blot, (capture) ELISA, a (micro)array, etcetera.

The invention provides as an exemplary embodiment a set of 81 reporter genes whose expression is correlated with the existence and stage of follicular lymphoma. This set of reporter gene is depicted in table 2. Each of said genes is differentially expressed in follicular lymphoma cells and surrounding non-malignant cells as compared to a healthy control of non-malignant B cells and surrounding non-malignant cells (non-malignant tonsillectomy specimens). The invention also provides a use of at least 5 of these markers for staging a follicular lymphoma. An expression profile of a sample comprising follicular lymphoma cells and/or non-malignant cells, preferably T cells, is determined. Preferably, said expression profile is compared with an expression profile of a control comprising non-malignant B cells and T cells. Most preferably, said expression profile is compared with an expression profile as depicted in FIG. 2B. An expression profile comparable to the upper part of FIG. 2B is indicative for an aggressive disease course while an expression profile comparable to the lower part of FIG. 2B is indicative for an indolent disease course. Similarity of expression profiles is for instance calculated using the Rosetta Error Model (Hughes T R, Marton M J, Jones A R et al. Functional discovery via a compendium of expression profiles. Cell. 2000; 102:109-126) and/or using the methods described on page 6 line 3-page 7 line 19 of WO 02/103320. In one embodiment a method of the invention is provided, comprising determining in a sample comprising said non-malignant cell the presence and/or amount of expression product of at least 5 reporter genes listed in table 2. More preferably, the presence and/or amount of expression product of at least 10 reporter genes listed in table 2 is determined. Even more preferably, the presence and/or amount of expression product of at least 30 reporter genes listed in table 2 is determined. Even more preferably, the presence and/or amount of expression product of at least 50 reporter genes listed in table 2 is determined. Most preferably, the presence and/or amount of expression product of each of the reporter genes listed in table 2 is determined. The more expression products are determined, the more reliable is the assay.

In one embodiment the invention provides a method for staging a follicular lymphoma present in a sample, said sample further comprising non-malignant cells, comprising detecting a difference in the expression of a plurality of genes of said follicular lymphoma and said non-malignant cells relative to a control, said plurality of genes consisting of at least 5 genes listed in table 2. In preferred embodiments said plurality of genes consists of at least 10, 15, 20, 25, 30, 35, 40, 45, 50, 55, 60, 65, 70, 75, 80 and/or 81 of the genes listed in table 2. The greater the amount of genes, the more reliable is the assay. In one embodiment said expression profile comprises the presence and/or amount of RNA, preferably mRNA, of said genes listed in table 2.

In a preferred embodiment said non-malignant cell and said malignant cell are capable of interacting. According to the invention, an expression profile of a malignant functional cell and a non-malignant cell which are capable of interacting is particularly indicative for the presence and the stage of said malignant functional cell. Preferably, said non-malignant cell is present in the surroundings of said malignant cell, such that interaction is enabled. A preferred example of a non-malignant cell of the invention is a T cell infiltrated into, or in close vicinity to, a lymphoma.

A malignant functional cell of the invention is preferably capable of producing an antibody, enzyme and/or hormone. In a preferred embodiment said malignant functional cell comprises a B cell. Said B cell is preferably still capable of responding to activation by T helper cells and capable of producing antibody. The presence of a malignant B cell which is still functional, such as a malignant follicular lymphoma cell, results in an expression pattern of said B cell and a non-malignant cell such as a T helper cell which is indicative for the presence and stage of said malignant B cell. In one aspect a method of the invention is provided wherein said non-malignant cell comprises a T cell, preferably a T helper cell. Preferably, an expression profile of a follicular lymphoma cell and a surrounding T helper cell is determined. In order to efficiently stimulate B cells and CTL, a T helper cell is stimulated by an antigen presenting cell. Antigen presenting cells are also in the vicinity of a lymphoma. According to the present invention, an expression profile of a malignant functional cell and a surrounding non-malignant cell is indicative for the presence and stage of said malignant functional cell. In one embodiment, said expression profile comprises an expression profile of a lymphoma cell and an antigen presenting cell such as a dendritic cell.

Now that the invention has provided the insight that an expression profile of a malignant functional cell and a non-malignant cell is indicative for the presence and/or stage of said malignant cell, it has become possible to establish gene-expression profiles capable of detecting and/or staging a wide variety of malignant functional cells. Establishment of a gene-expression profile of 81 genes capable of detecting and staging follicular lymphoma is described in the examples. Likewise, an expression profile capable of detecting and/or staging another kind of malignant functional cells is established. Said expression profile is established by measuring an expression profile of said malignant functional cell and/or a non-malignant cell. Said non-malignant cell is possibly surrounding said malignant cell, such that interaction is possible. Preferably, an expression profile of a sample comprising said malignant functional cell and a non-malignant cell is determined and compared to a reference and/or control expression pattern. In one preferred embodiment said malignant functional cell comprises a mucosa-associated lymphoid tissue lymphoma cell. In another preferred embodiment said malignant functional cell comprises a renal cell carcinoma (such as a Grawitz tumor) and/or a prostate cancer cell. A set of reporter genes whose expression is correlated with the existence and stage of mucosa-associated lymphoid tissue lymphoma cell, renal cell carcinoma (such as a Grawitz tumor) and/or a prostate cancer cell is also herewith provided. Said sets are established using a comparable approach as outlined in the examples for follicular lymphoma. Contrary to prior art approaches, the use of malignant cells only for establishing an expression profile is avoided. Instead, a sample comprising non-malignant cells is used. Preferably, said non-malignant cells comprise cells capable of interacting with said malignant cells. More preferably, said non-malignant cells comprise cells surrounding said malignant cells, such as infiltrating T cells.

Reporter genes of the invention and their expression products are suitable drug targets. Expression of said reporter genes is influenced in order to treat malignant functional cells. For instance, expression of a reporter gene overexpressed in aggressive malignancy stages is at least in part reduced. This way aggressive malignant cells are at least in part counteracted, and/or conversion of a malignant functional cell from an indolent stage to an aggressive stage is counteracted. Preferably, a medicament is administered to an individual suffering from malignant functional cells which medicament is capable of at least in part counteracting expression of a reporter gene normally overexpressed in aggressive malignancy stages. In one embodiment a medicament is administered to an individual suffering from malignant functional cells which medicament is capable of at least in part enhancing expression of a reporter gene normally underexpressed in aggressive malignancy stages. In a further embodiment, a medicament is used that is capable of at least in part counteracting at least one function of an expression product of a reporter gene. For instance, a compound capable of specifically binding a protein encoded by said overexpressed reporter gene is suitable for counteracting said malignant functional cells. Binding of said compound to said protein at least in part inhibits a function of said protein. Said compound preferably comprises an antibody, single chain variable fragment and/or a Fab fragment. In one embodiment, said protein is associated to the surface of a malignant functional cell, since a drug directed to a surface-associated protein can more easily reach its target.

The invention furthermore provides an array comprising a compound capable of specifically binding an expression product of a reporter gene of the present invention. Said compound (probe or oligonucleotide probe) preferably comprises a nucleic acid sequence capable of specifically hybridizing to an RNA product of a reporter gene of the invention. Said nucleic acid sequence is preferably complementary to at least part of a reporter gene of the invention, said part being at least long enough for being specific for said particular reporter gene. Said part is preferably at least 30 base pairs long, more preferably at least 50 base pairs long, more preferably at least 70 base pairs long, more preferably at least 100 base pairs long, more preferably at least 150 base pairs long, most preferably at least 200 base pairs long. An increasing length contributes to the specificity of an assay performed with said array. In one embodiment an array comprising nucleic acid molecules capable of specifically binding to (an expression product of) at least 5 reporter genes listed in table 2 is provided. Preferably, said array comprises nucleic acid molecules capable of specifically binding to at least 10, more preferably 15, more preferably 20, more preferably 25, more preferably 30, more preferably 35, more preferably 40, more preferably 45, more preferably 50, more preferably 55, more preferably 60, more preferably 65, more preferably 70, more preferably 75, more preferably 80 (expression products of) reporter genes listed in table 2. Most preferably an array is provided comprising nucleic acid molecules capable of specifically binding to (expression products of) each of the reporter genes listed in table 2. The greater the amount of reporter genes, the more reliable an assay performed with said array is. Said expression products preferably comprise mRNA. In a preferred embodiment an array of the invention is a microarray.

Nucleic acid molecules or probes specific for a reporter gene listed in table can all be used in an array or a method of the invention. Some reporter genes of table 2 are more discriminating than others. In table 3 reporter genes are sorted for their discriminating power. The column identified as classifier order indicates the order of discrimination power. Reporter genes are listed such that a lower classifier number indicates a higher discriminating power. Thus in a preferred embodiment an array of the invention comprises at least one probe specific for a reporter gene of rows or classifier order 1-5 of table 3 (i.e. specific for a reporter gene with genbank accession number AA428607, AA074535, AA485732/AI732826, AA448664 or R63543). Preferably said array comprises probes specific for the reporter genes of rows or classifier order 1-5 of table 3. In another preferred embodiment an array of the invention comprises at least one probe specific for a reporter gene of rows or classifier order 6-10, 11-15, 16-20, 21-25, 26-30 or 31-35 of table 3. In a preferred embodiment said array comprises at least 20 nucleic acids molecules or probes specific for reporter genes listed in table 2, wherein at least 1 of said nucleic acids molecules or probes specific for reporter genes is a reporter gene of rows or classifier order 1-5 of table 3. Preferably, said at least 20 nucleic acid molecules or probes comprise 5 nucleic acids molecules or probes specific for reporter genes is a reporter gene of rows or classifier order 1-5 of table 3. FIG. 5 depicts the performance of arrays of the invention on samples comprising expression products obtained from tumor biopsies of various individuals suffering from follicular lymphomas. It can be seen that an array of the invention perform well with as few as 5 nucleic acid molecules or probes specific for reporter genes listed in table 2. The performance increases with increasing number of said molecules or probes for reporter genes of table 2. In a preferred embodiment an array of the invention therefore comprises at least 20 and more preferably at least 30 nucleic acid molecules or probes specific reporter genes listed in table 2. Best performance is obtained when said array comprises 60 or more nucleic acid molecules or probes specific reporter genes listed in table 2, thus in a particularly preferred embodiment an array of the invention comprises at least 60 nucleic acid molecules or probes specific reporter genes listed in table 2.

A probe for a nucleic acid expression product of a reporter gene of table 2 is typically a nucleic acid probe. However, currently many different analogues to nucleic acid probes are being used. Such analogues include but are not limited to peptide nucleic acid (PNA). Though at present not economically competitive with “normal” nucleic acid, such analogue can of course also be used as probe or molecule specific for an expression product of a reporter in an array and or method of the invention.

An array of the invention preferably further comprises a reference probe. Reference probes are useful for a variety of purposes. Reference probes can for instance advantageously used for normalization purposes. Sometimes variations in the profile intensity occur, or systematic variation in the pattern occurs in a test or sample correlated way. This can for instance be due to variations in the taking of the biopsy or the manipulation of the sample to extract or prepare the expression product(s). Such variations are noted by using reference probes. Depending on the kind of variation, tests may be discarded or corrected for by using the signal(s) obtained from the reference probes in the array as a guidance. In a preferred embodiment an array comprises between 50 and 20.000 reference probes. In a particularly preferred embodiment said array comprises between 100 and 5000 reference probes. This range of probes is typically more than enough for detection of mentioned variations.

The invention further provides a method for determining whether a sample comprises a malignant functional cell comprising providing an array of the invention with expression products from said sample and determining whether an expression profile of said sample on said array is indicative for the presence and/or stage of said malignant functional cell. In a preferred embodiment said malignant functional cell is present in a tumor biopsy from an individual suffering from Follicular lymphoma. The invention further provides the use of an array of the invention for determining whether a sample of an individual comprises Follicular lymphoma cells. The invention also provides the use of an array of the invention for determining the stage or grade of Follicular lymphoma that an individual has from which the tumor biopsy was obtained.

In one embodiment at least 50% of the probes on an array of the invention is capable of specifically hybridizing with (an expression product of) a reporter gene listed in table 2. In preferred embodiments, at least 60%, 65%, 70%, 75%, 80%, 85%, 90%, 93%, 95%, 97%, 98%, 99% or 100% of the probes on an array of the invention is capable of specifically hybridizing with a reporter gene listed in table 2. The greater the amount of probes capable of specifically hybridizing with (an expression product of) a reporter gene listed in table 2, the more reliable is the assay performed with said array.

In another preferred embodiment an array comprising a compound capable of specifically binding an expression product of a reporter gene of the present invention is provided, wherein said compound is capable of specifically binding at least part of a protein encoded by a reporter gene of the invention. Said compound preferably comprises an antibody, single chain variable fragment and/or a Fab fragment. In one embodiment an array comprising compounds capable of specifically binding proteinaceous molecules encoded by at least 5 reporter genes listed in table 2 is provided. Preferably, said array comprises compounds capable of specifically binding proteinaceous molecules encoded by at least 10, 15, 20, 25, 30, 35, 40, 45, 50, 55, 60, 65, 70, 75, 80 or 81 reporter genes listed in table 2. The greater the amount of reporter genes, the more reliable an assay performed with said array is.

In one embodiment at least 50% of the compounds on an array of the invention is capable of specifically binding proteinaceous molecules encoded by a reporter gene listed in table 2. In preferred embodiments, at least 60%, 65%, 70%, 75%, 80%, 85%, 90%, 93%, 95%, 97%, 98%, 99% or 100% of the compounds on an array of the invention is capable of specifically binding proteinaceous molecules encoded by a reporter gene listed in table 2. The greater the amount of compounds, the more reliable is an assay performed with said array.

The invention further provides an array for detecting and/or staging a malignant functional cell, comprising a preferably positionally-addressable array of polynucleotide probes bound to a support, said polynucleotide probes comprising a sequence complementary and hybridizable to at least part of at least 5 reporter genes listed in table 2, said part being at least long enough for being specific for said particular reporter gene. Said part is preferably at least 30 base pairs long, more preferably at least 50 base pairs long, more preferably at least 70 base pairs long, more preferably at least 100 base pairs long, more preferably at least 150 base pairs long, most preferably at least 200 base pairs long. An increasing length contributes to the specificity of an assay performed with said array. An array of the invention preferably comprises a positionally-addressable array of polynucleotide probes bound to a support, said polynucleotide probes comprising a sequence complementary and hybridizable to at least part of at least 10, 15, 20, 25, 30, 35, 40, 45, 50, 55, 60, 65, 70, 75, 80, or 81 reporter genes listed in table 2. Preferably, at least 50% of the probes on the array is complementary to at least part of the reporter genes listed in table 2. Most preferably, at least 60%, 65%, 70%, 75%, 80%, 85%, 90%, 93%, 95%, 97%, 98%, 99% or 100% of the probes on the array is complementary to at least part of the reporter genes listed in table 2. In one embodiment an array is provided comprising at least one probe comprising at least part of a sequence as depicted in Table 2, said part being at least long enough for being specific for said sequence. Said part is preferably at least 30 base pairs long, more preferably at least 50 base pairs long, more preferably at least 70 base pairs long, more preferably at least 100 base pairs long, most preferably at least 150 base pairs long.

An array of the invention preferably comprises a microarray. An array of the invention is generated using known methods in the art such as for instance described on page 121 line 33-page 122 line 19 of WO 02/103320 and/or according to Brazma A et al, Minimum information about a microarray experiment (MIAME)-toward standards for microarray data. Nat Genet. 2001; 29:365-371.

An array of the present invention is particularly suitable for detecting and/or staging a malignant functional cell. In one embodiment a sample comprising said malignant cell together with non-malignant cells is brought into contact with an array of the invention. In one embodiment said sample is processed before it is brought into contact with an array of the invention. For instance, nucleic acid and/or protein is purified from said sample using known methods such as for instance the RNA purification protocol described in the examples, and/or cell lysis, centrifugation, chromatography and/or desalting (protein purification). After incubation of an array of the invention with (a purified part of) said sample, non-bound components are preferably washed away. Subsequently it is determined whether a binding pattern is obtained which is indicative for the presence and/or stage of said malignant functional cells. Binding of expression products of reporter genes is detected using known methods in the art, such as (radioactive or fluorescent) labeling of expression product. Since many methods for purifying and detecting expression products are available, a method of the present invention can be performed in many alternative ways.

In one aspect the invention therefore provides a method for detecting and/or staging a malignant functional cell comprising:

providing an array of the present invention with a sample comprising expression products from said malignant functional cell and from a non-malignant cell; and

determining whether an expression profile indicative for the presence and/or stage of said malignant functional cell has bound to said array.

Preferably said expression product comprises nucleic acid. In another preferred embodiment said expression product comprises protein. In one embodiment said malignant functional cell comprises a follicular lymphoma cell, a mucosa-associated lymphoid tissue lymphoma cell, a cell of a Grawitz tumour and/or a prostate cancer cell.

The invention has provided the insight that an expression profile of malignant functional cells and non-malignant cells is indicative for the presence and/or stage of said malignant functional cells. Said malignant cell and said non-malignant cell are preferably capable of interacting, at least at some stages of the disease. Without being bound to theory, it is believed that interaction between a malignant functional cell and another kind of cell influences both functioning of said malignant cell and functioning of said non-malignant cell. As a result, the expression profile of said malignant cell and said non-malignant cell changes. For instance, continuous stimulation by T helper cells enhances follicular lymphoma development. An expression profile of a sample comprising follicular lymphoma cells and T helper cells is indicative for the presence and/or stage of follicular lymphoma. Since a malignant functional cell of the invention is often still capable of interacting with other kind of cells, intervention between said interaction at least in part counteracts a disease involving a malignant functional cell of the invention.

The invention therefore provides a method for treating a disease involving a malignant functional cell, comprising at least in part inhibiting an interaction between said malignant functional cell and a non-malignant cell. Preferably, signaling of a T cell is at least in part counteracted. Said T cell preferably comprises a T helper cell. Signaling of a T cell is defined as influencing at least one characteristic of a malignant functional cell of the invention by said T cell. Said characteristic for instance comprises replication and/or the production of antibodies. Signaling of a T cell for instance comprises secretion of cytokines.

Said interaction between said malignant functional cell and said non-malignant cell is for instance at least in part inhibited by a compound capable of binding said malignant cell, said non-malignant cell, and/or a compound involved with said interaction such as for instance a cytokine. Said compound for instance comprises an antibody, single chain variable fragment and/or a Fab fragment. Administration of said compound at least in part inhibits proliferation of a malignant cell and, hence, is used as a medicament. The invention thus provides a compound capable of at least in part inhibiting signaling of a T cell for use as a medicament.

Said compound is suitable for treating a disease involving a malignant functional cell. A use of a compound capable of at least in part inhibiting signaling of a T cell for the preparation of a medicament for treating a disease involving a malignant functional cell is therefore also herewith provided.

In one embodiment said disease comprises follicular lymphoma, mucosa-associated lymphoid tissue lymphoma, a Grawitz tumour and/or prostate cancer. Said compound is preferably capable of at least in part counteracting an interaction between a malignant B cell and a T helper cell. Non limiting examples of suitable compounds are CXCR-4 inhibitors and fludarabin.

### DETAILED DESCRIPTION

One kind of malignancy-related disease which involves staging problems is follicular lymphoma. Follicular lymphomas (FL) form a well defined clinicopathological entity and are the second most frequent lymphoma in adults. In the Netherlands, approximately 2000 new cases of non-Hodgkin's lymphoma are seen yearly, of which 20% are FL.

The t(14; 18) translocation lies at the basis of oncogenesis in follicular lymphoma. Follicular lymphoma is a disease characterized by a long clinical course for most patients and marked by frequent relapses that vary in clinical aggressiveness over time. The median overall survival varies from 6-12 years, but the clinical course of this disease is highly variable. Some patients have waxing and waning disease for several years without any treatment. Generally, the malignancy is chemo- and radiosensitive with remission rates of 70-80%. However, the disease inevitably relapses and curative treatment is at present not possible.

Transformation to diffuse large B-cell lymphoma (DLBCL) is a common event and occurs in approximately 80-85% of the patients. Primary treatment may vary from watchful waiting to high dose chemotherapy with stem cell transplantation with the aim to reach long lasting disease-free survival and in a small subset of patients possibly cure. At subsequent relapse, treatment is again stratified for clinical aggressiveness. Transformation and development of unresponsiveness to chemotherapy in the course of the disease form the main causes of death in patients with FL and in these situations more aggressive and possibly experimental therapy is justified. In individual patients and at each relapse the diagnosis of the phase of the disease (indolent or aggressive) is important for the choice of optimal therapy. Timing of the most optimal treatment is of particular importance for the benefit of patients, since both over-treatment and under-treatment in relation to the clinical aggressiveness of the disease affects event-free survival and overall survival. Unnecessary or premature use of anthracyclin is harmful with respect to quality of life and morbidity, and the use is limited by an absolute maximum tolerable dose. Conversely, withholding aggressive chemotherapy (in clinically aggressive disease) will result in insufficient therapy response with consequences for disease-free and overall survival, especially in the first line.

Thus far, morphological sub-classification has been used as a major guide in the choice of therapy in FL. In the World Health Organization Classification (WHO), FL is graded as 1, 2, 3a and 3b according to the number of large transformed cells (centroblasts) per high-power field (Nathwani B N, Harris N L, Weisenburger D et al. In: Jaffe, E. S.; Harris, N. L.; Stein, H.; Vardiman, J. W., eds. Pathology and genetics of tumours of haematopoietic and lymphoid tissues. Lyon: IARC Press; 2001). Even though these criteria appear objective, grading has a notoriously poor reproducibility among community pathologists, as well as experienced hematopathologists (agreement 61-73%). This is due to the subjective nature of morphological grading and the inherent inadequacy of the criteria set for “transformed cells”. The cellular morphology of transformed cells may be highly variable and the component of transformed cells may be heterogeneous within the biopsy. Therefore, classical morphological grading results in too many inconclusive cases (10 to 30% of all cases) and is not optimally suitable as a guideline in the choice of therapy in daily practice. An alternative method with objective and reproducible parameters to judge clinical aggressiveness at any decision point for treatment in patients with FL is therefore desired. Clinical prognostic indices, such as the International Prognostic Index (IPI) (A predictive model for aggressive non-Hodgkin's lymphoma. The International Non-Hodgkin's Lymphoma Prognostic Factors Project. N Engl J Med. 1993; 329:987-994; Hermans J et al, Blood. 1995; 86:1460-1463) and variations that are more tailored to FL (FLIPI) (Federico M et al, Intergruppo Italiano Linfomi. Blood. 2000; 95:783-789; Solal-Celigny P and Roy P. Ann Oncol. 2002; 13: Abstract 54) are in principle suitable markers of clinical course. However, the discriminative value for the majority of patients is limited since most patients will fall in the low and low-intermediate risk groups due to the limitations and resolution of the system. Moreover, these indices are only formally validated for predictive value at diagnosis. Hence, other means of stratification are desired.

The invention is further illustrated by the following examples. The examples do not limit the scope of the invention in any way.

### BRIEF DESCRIPTION OF THE DRAWINGS

FIG. 1. Unsupervised clustering of 72 FL samples, grades 1, 2 and 3, shows a relative biological homogeneity of FL. There is a separation in three main groups with no enrichment for morphological grades or clinical behavior in either of the groups. Each row represents a malignancy and each column a single gene. Gene-expression is depicted according to the color-bar. Red indicates a high level of mRNA-expression relative to the reference and green indicates a low level of expression. Selected morphological and clinical data are depicted in the right panel. For clinical features: white indicates indolent; black indicates aggressive disease behavior (see Table 1 for criteria). For morphological grading: white indicates grade 1, dashed grade 2, black grade 3. For IPI: white denotes scores 0, 1 or 2, black 3, 4 or 5. Gray denotes insufficient data.

FIG. 2. Supervised classification of 12 paired lymphoma samples (training series).

A. Correlation plot of the FL-stratification profile. Correlation of the expression profile of each malignancy sample with the average expression profile of all “indolent” samples is depicted on the vertical axis, and the correlation of the expression profile of each malignancy with the average expression profile of all “aggressive” malignancy samples is shown on the horizontal axis. Malignancies classified above the threshold are classified as molecular high-grade; malignancies below the threshold are classified as molecular low-grade.

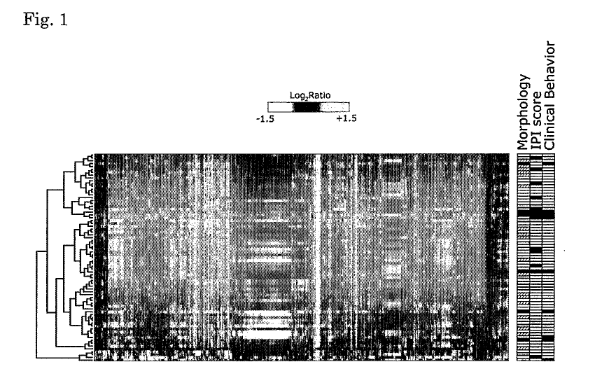

B. Expression data matrix of 81 marker genes from malignancies of the indolent as well as the aggressive phase of 12 follicular lymphoma patients. Each row represents a malignancy and each column a gene. Genes are ordered on the basis of their SNR. Maliganncies are rank-ordered according to the difference in correlation with the average “high-grade profile” and the correlation with the “low-grade profile” (middle panel). The solid yellow line is the classifier with optimal accuracy; above the yellow line patients have an aggressive disease course, below the yellow line, patients have an indolent disease course at the disease episode at the time of biopsy. Selected clinical data is shown in the right panel: Morphological data, white indicates FL grade 1 or 2; black indicates FL grade 3B or DLBCL; IPI scores and clinical behavior at time of biopsy as in FIG. 1.

FIG. 3. Correlation and pattern of expression of genes used to determine the clinical characteristics of 58 lymphoma samples (independent validation series).

A. Correlation plot of the FL-stratification profile. Correlation of the expression profile of each malignancy sample with the average expression profile of all “low-grade” samples of the training series is depicted on the vertical-axis, and the correlation of the expression profile of each malignancy with the average expression profile of all “high-grade” malignancy samples of the training series is shown on the horizontal-axis. Malignancies classified above the threshold are classified as molecular high-grade; malignancies below the threshold are classified as molecular low-grade.

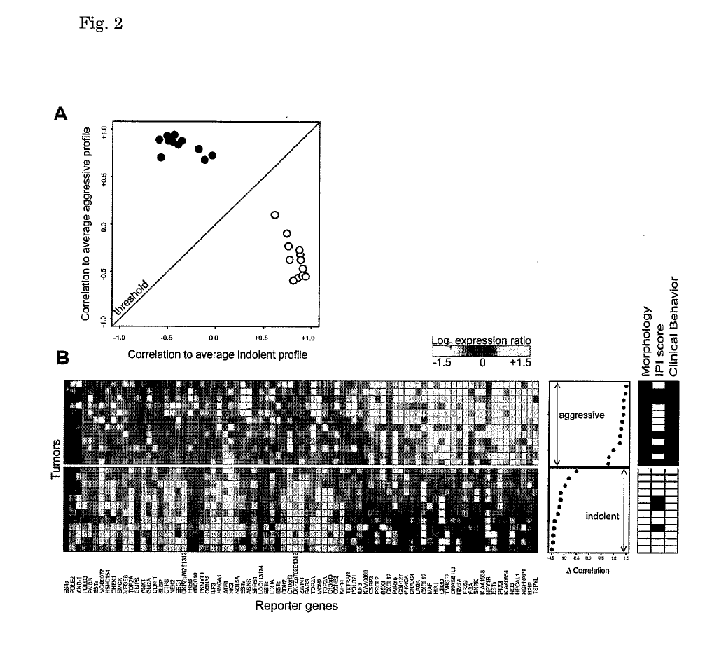

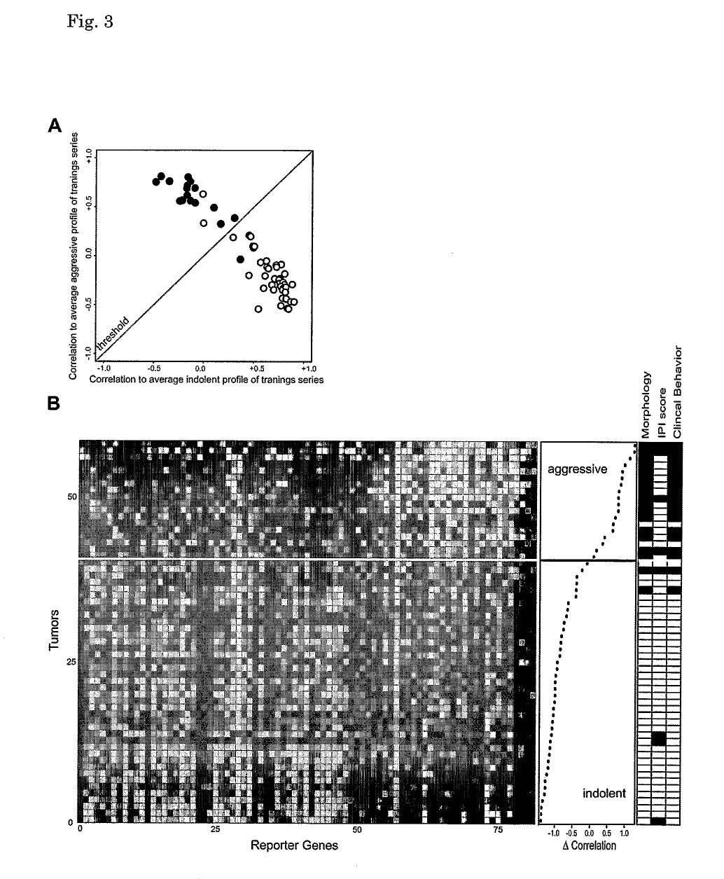

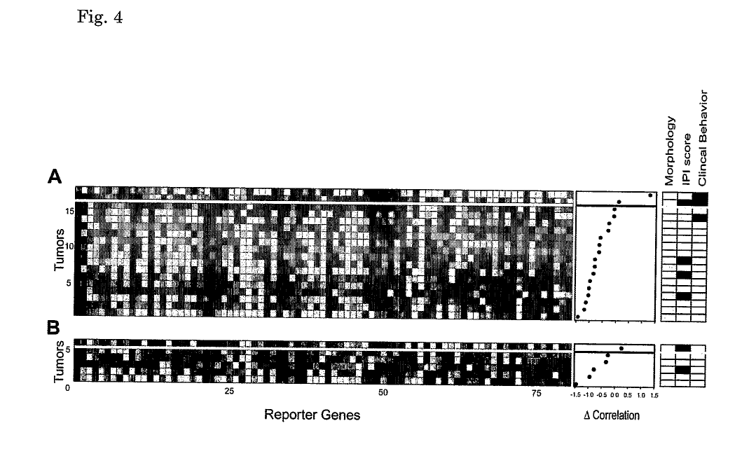

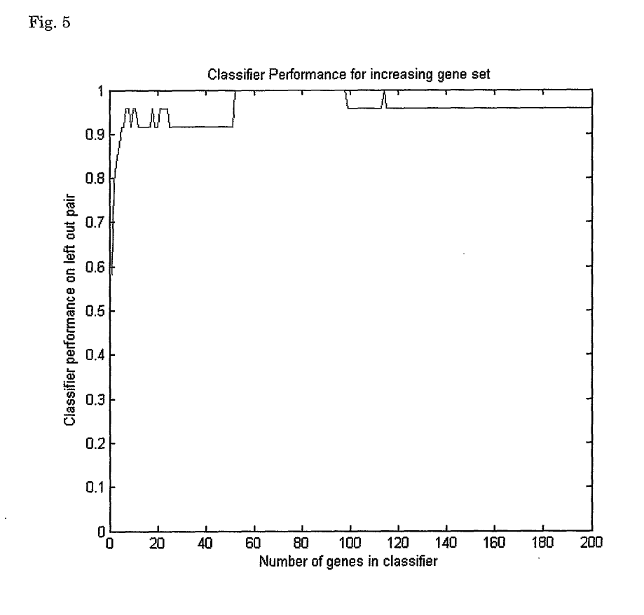

B. Expression data matrix of 81 marker genes from malignancies of the indolent as well as the aggressive phase of follicular lymphoma patients. Each row represents the FL-stratification profile for one malignancy. Each column represents the relative expression of one gene. The genes in the horizontal direction are arrayed in the same order as in FIG. 2B. Malignancies are rank ordered according to the difference in correlation with the average “aggressive” profile and the correlation with the “low-grade profile” of the training set (middle panel). The yellow line is the threshold as determined in FIG. 2 (see FIG. 2 for color scheme). In the panel on the right morphological and clinical features are shown of the samples similar to features shown in FIG. 2B. Of the 58 samples, 4 were misclassified (93% accuracy).

FIG. 4. Pattern of gene expression of genes used to determine the clinical characteristics of 24 Follicular Lymphoma samples (test series). Panel A shows the expression data matrix of 18 malignancy samples with an unclear morphology across the 81 gene profile. The genes in the horizontal direction are arrayed in the same order as in FIG. 2B. Malignancies are rank ordered according to the difference in correlation to the molecular high-grade and low-grade profile as in FIG. 2 (middle panel). The yellow line is the threshold as determined in FIG. 2B (see FIG. 2 for color scheme). Morphological data, IPI scores and Clinical behavior are shown on the right. White, gray and black as indicated in FIG. 1B. Of the 18 samples, the classifier predicted the actual clinical behavior correctly in 17 patients (accuracy 94%). Panel B shows expression data of six malignancy samples with unequivocal morphological and clinical indolent disease, but who presented with full-blown aggressive disease within 10 months. One sample was classified as molecular high-grade.

FIG. 5. Classifier performance for increasing gene set.

This figure presents a measure for the performance of the nucleic acid set depicted in table 2 and table 3. It can be seen that tests using arrays of the present invention already perform well when they contain as few as 5 nucleic acids molecules or probes of table 2 or table 3. Performance peaks when 60 or more genes are included in the set. More than 100 genes in the set again decreases the performance of the test as with increasing numbers, nucleic acids molecules or probes are incorporated for expression products that have limited or no discriminative power.

### EXAMPLES

### Example 1

Methods

Design of the Study

The aim of this study was to search for a gene-expression profile that assesses indolent versus aggressive clinical behavior both at diagnosis and at relapse for the disease episode at the moment of biopsy to replace current inadequate morphological and clinical methods. Since adequate upfront criteria and gold standards for clinical behavior are not available, the complete course of the disease was evaluated retrospectively for all patients and each disease episode was defined as either indolent (non-aggressive) or aggressive. To qualify as a clinically non-aggressive or indolent disease episode, the following criteria had to be met: 1) absence of B symptoms, elevated LDH, rapid generalized disease progression in the preceding 3 months, and 2) if treated, a good malignancy response (partial or complete remission) after at least one of two non-anthracyclin-containing, non-high dose chemotherapy regimens (e.g. chlorambucil, CVP, or fludarabin). An aggressive disease episode was defined as development of B-symptoms and LDH and/or rapid generalized disease progression within the preceding 3 months, and/or progressive disease during successive treatment with two or more non-anthracyclin, non-high dose chemotherapy regimens (Table 1). In case of ambiguous clinical situations, the patient was not included in this study. Since FL is a genetically very homogeneous disease and the variation in expression patterns is within small limits, a supervised approach was chosen. A gene-expression profile was developed in a set of paired samples and validated in a separate series of patients. Most importantly, the profile was tested in a third series of cases with ambiguous morphological features.

Patient Selection

Malignancy samples from patients with primary nodal follicular lymphoma treated between 1984 and 2002 were selected from the fresh-frozen-tissue banks of the Netherlands Cancer Institute, Central Laboratories Friesland, University Medical Center Nijmegen, Leiden University Medical Center and Groningen University Medical Center according to the following criteria: availability of a representative frozen sample and paraffin-embedded tissue, proven diagnosis of FL and availability of complete clinical data at presentation and during follow-up. The study was approved by the medical-ethics committee of the Netherlands Cancer Institute.

106 samples from 80 patients were included in this study. Of these patients, the full medical history from diagnosis to last follow-up or death was evaluated retrospectively including data on IPI parameters (A predictive model for aggressive non-Hodgkin's lymphoma. The International Non-Hodgkin's Lymphoma Prognostic Factors Project. N Engl J Med. 1993; 329:987-994), treatment and treatment results. All relevant biopsy material was reviewed, including full immunohistochemical workup (CD20, CD3, CD5, bcl-2, bcl-6, CD10, CD21) by three hematopathologists (DdJ, RK, JvK), classified and graded according to the WHO classification. A selection of clinical and histological data is summarized in Supplementary Table 1. IPI parameters are grouped in a low/low-intermediate risk group for IPI score 0, 1 or 2, and a high-intermediate/high-risk group for IPI score 3, 4 or 5. For each patient indolent and aggressive disease episodes were assigned in retrospect according to the criteria described in the design of the study. It should be noted that for the retrospective clinical classification histological grading was not included. All patients for whom due to lack of data the clinical episode of the available biopsy sample could not be reliably assigned to the indolent or aggressive phase of the disease, were excluded.

Treatment varied and was administered according to local protocols at the time of diagnosis, including cyclophosphomide/vincristine/prednisone (CVP), fludarabin, chlorambucil, chlorambucil/prednisone, and cyclophosphamide/doxorubicin/vincristine/prednisone (CHOP-like) with or without radiotherapy. Different “indolent-type” treatment protocols have been shown to be equally effective in FL and do not result in different disease-free and overall survival (Horning S J. Follicular lymphoma: have we made any progress? Ann Oncol. 2000; 11 Suppl 1:23-27; Horning S J. Natural history of and therapy for the indolent non-Hodgkin's lymphomas. Semin Oncol. 1993; 20:75-88). Since the aim of the study is to find a profile related to clinical behavior at the time of biopsy and not a survival-related or treatment-response related profile, variability in treatment will not influence our study results.

The patient samples were divided in three different groups.

1) Training series: Patients of whom paired samples corresponding to the concordant clinical/morphological indolent and to concordant clinical/morphological aggressive episodes were available; i.e., combined indolent disease course and FL grade 1 or 2 versus aggressive episode and FL grade 3B or DLBCL (Nathwani B N et al. In: Jaffe, E. S.; Harris, N. L.; Stein, H.; Vardiman, J. W., eds. Pathology and genetics of tumours of haematopoietic and lymphoid tissues. Lyon: IARC Press; 2001). Twenty-four paired samples of both phases in 12 patients were used to build the classifier profile and to minimize patient specific variation. Age at diagnosis of these selected patients ranged from 24 to 74 years (median 49.5 years, median follow up 80 months, range 29-304 months). Transformation to aggressive disease occurred with a median interval of 64 months (range 22-288 months).

2) Validation series: 58 independent samples (54 patients) of clinical/morphological indolent phases or clinical/morphological aggressive phases. Age at diagnosis ranged from 27 to 78 years (median 53 years). The median duration of follow-up was 71 months (range 4 to 206 months). The aggressive phase samples consisted of 18 patient samples, median interval to transformation 36.5 months, range 3-139 months. The samples of the indolent group consisted of 18 patient samples (median follow-up 91 months, range 24-68 months) as well as 22 samples from the indolent phases previous to transformation (median interval to transformation 47 months, range 21-168 months).

3) Validation (series) for difficult cases: Eighteen patient samples were selected that showed ambiguous morphological features according to the review panel (DdJ, RK, HvK) and were scored as inconclusive in the past: borderline between FL grade 2 and grade 3A or a histology characterized by the dominant presence of small centroblast-like cells that fell outside the consensus criteria for classical “large transformed cells” ((Nathwani B N et al. In: Jaffe, E. S.; Harris, N. L.; Stein, H.; Vardiman, J. W., eds. Pathology and genetics of tumours of haematopoietic and lymphoid tissues. Lyon: IARC Press; 2001).

In addition, 6 patients were selected who presented with a FL grade 1 and clinically indolent behavior in the first biopsy, but showed histological transformation to DLBCL and progression to clinically high-grade and aggressive disease within 10 months (median time to progression 6.5 months, range 3 to 10 months).

RNA Isolation, Amplification and Labeling and Hybridization:

Detailed protocols for RNA isolation, amplification, labeling and hybridization can be found at http://www.nki.nl/nkidep/pa/microarray and are presented above. All samples were co-hybridized with a standard reference of pooled and amplified RNA isolated from tonsillectomy specimens of 5 patients who underwent routine tonsillectomy for chronic ear/nose/throat-infections. This tissue reference was chosen to provide a lymphoid reference containing all genes that are potentially expressed in the malignancy tissues at a significant level and biologically closely related to the malignancy samples to be able to identify small changes in expression levels between the malignancy groups.

Microarray Slides:

Microarray slides were prepared at the central microarray facility (CMF) at the Netherlands Cancer Institute. Sequence verified cDNA clones (InVitrogen, Huntsville, Ala.) were spotted onto poly-L-lysine-coated glass slides using the Microgrid II arrayer (Apogent, Cambridge, UK) with a complexity of 19,200 spots per slide. A complete list of genes and controls included on the slides is available on the CMF web site (http://microarrays.nki.nl), as well as details on the process of preparing the DNA for spotting and preparation of the slides.

Normalization:

Fluorescent intensities were normalized and corrected for a variety of biases that affect the intensity measurements (e.g., color bias and print tip bias) according to Yang et al. (Yang Y H, Dudoit S, Luu P et al. Normalization for cDNA microarray data: a robust composite method addressing single and multiple slide systematic variation. Nucleic Acids Res. 2002; 30:e15). Weighted averages, and confidence levels were computed according to the Rosetta Error Model (Hughes T R, Marton M J, Jones A R et al. Functional discovery via a compendium of expression profiles. Cell. 2000; 102:109-126).

Unsupervised Clustering:

Gene clustering and malignancy clustering were performed independently using agglomerative hierarchical clustering in the software program genesis (Sturn A, Quackenbush J, Trajanoski Z. Genesis: cluster analysis of microarray data. Bioinformatics. 2002; 18:207-208). For gene clustering, complete linkage similarity metrics among genes were calculated on the basis of expression ratio measurement across all malignancies. Similarly, for malignancy clustering complete linkage clustering was calculated based on expression ratio measurements across all significant genes.

Supervised Classification:

To reliably discriminate between the indolent and aggressive malignancies, the following supervised classification method was used to build the FL-stratification classifier:

1) From the 19,200 genes on the microarray, genes that were significantly different from the reference in at least two malignancies, were selected (significance was based on the P-values (p<0.01) computed with the Rosetta Error Model (Hughes T R, Marton M J, Jones A R et al. Functional discovery via a compendium of expression profiles. Cell. 2000; 102:109-126)).

2) Calculation of the paired signal-to-noise-ratio (SNR) for each of the selected genes, and ranking of the genes (top ranked genes being genes that are best suited to separate indolent from aggressive samples).

3) Determination of top ranked genes that separate the two classes best when employed in a nearest prototype classifier (van't Veer L J, Dai H, van de Vijver M J et al. Gene expression profiling predicts clinical outcome of breast cancer. Nature. 2002; 415:530-536). Steps 2 and 3 are performed in a cross-validation procedure, where at each cross-validation iteration a matched pair is left out and employed to test the performance of the classifier trained on the remaining pairs.

The optimal number of genes that could separate molecular low-grade from molecular high-grade disease was determined in a leave-pair-out-cross-validation method. More specifically, in 12 repetitive steps, each paired sample was left out once, and for the remaining 22 samples, genes were ranked on the SNR. Then starting with the four most informative genes the classifier was trained on the 22 samples and employed to predict the outcome of the left-out pair. The classification of the left-out pair was predicted based on the largest Pearson correlation of the expression profile of each of the left-out samples with the mean expression levels of the remaining samples from the “indolent” or the “aggressive” patient samples for the selected reporter genes. Subsequently, the whole procedure was repeated by adding one gene at a time. Note that in order to avoid selection bias (Simon R et al, J Natl Cancer Inst. 2003; 95:14-18; Ambroise C et al, Proc Natl Acad Sci USA. 2002; 99:6562-6566), i.e. underestimation of the error rate, samples that were left out were not involved in any of the reporter selection steps.

The performance was measured as the average of the false positive and negative rates of the left-out samples. No further increase in performance was observed when the number of included reporter genes exceeded 81. Since the ranking in every cross-validation iteration produces a slightly different set of marker genes, the final set of 81 classifier genes in the FL-stratification classifier was determined by taking the 81 genes that occurred most frequently in each of the 12 steps.

Additional microarray information. The description of this study followed the MIAMI guidelines issued by the Microarray Gene Expression Data group (Brazma A, Hingamp P, Quackenbush J et al. Minimum information about a microarray experiment (MIAME)-toward standards for microarray data. Nat Genet. 2001; 29:365-371).

Results:

To provide an accurate and clinically relevant classification of FL, 106 specimens from 80 patients (supplementary Table 1) were analyzed for gene-expression profiles using 19K cDNA microarrays.

As a first step, unsupervised two dimensional hierarchical clustering was performed on 72 FL grade 1, 2, 3a and 3b samples to group genes on the basis of similarity across all malignancies and to group the malignancy samples according to similarities in gene expression (FIG. 1). This shows a relative homogeneity of FL as a single disease entity and shows a division in three main groups with no enrichment for morphological grade or clinical behavior. It should be noted, that multiples samples of single patients do not dominantly cluster together and both IPI-score and treatment are no component in the clustering. Unsupervised clustering is by far insufficient for clinical use, therefore, a supervised classification approach was chosen.

Development of a Molecular Profile for Low-Grade and High-Grade Disease in FL on the Basis of Paired Samples

To build a classifier a group of 24-paired samples from 12 patients was used for whom samples of both the clinically and morphologically indolent phase as well as the clinically and morphologically aggressive phase of the disease were available. Using the Rosetta Error Model (Hughes T R et al. Cell. 2000; 102:109-126), a total of 4760 mRNAs were found to be differentially expressed compared to the reference RNA (P-value<0.01 in 2 or more samples). From these, a set of 81 genes emerged from the cross-validation procedure with the optimal classification performance of 100%.

The correlation coefficients for each of the malignancy samples with the average expression of these 81 genes in either “low-grade” or “high grade” samples in the cross-validation were calculated and are shown in FIG. 2a. Malignancies positioned above the threshold were classified as aggressive; malignancies below the threshold were classified as low-grade. The line of equal correlation was chosen as threshold since misclassification in both directions (false positives and false negatives) would result in equally adverse treatment consequences for the patient.

The expression pattern of the 81 genes in the 24 learning samples is shown in the color plot of FIG. 2b, where the malignancies are ranked according to the difference in correlation with the average “high-grade” profile and the correlation with the “low-grade profile” (middle panel).

Expression-data were associated with data on clinical and histological parameters (histological grading, IPI score and clinical classification (FIG. 2b, right panel) and correlated perfectly with histological grading and clinical behavior. This is not unexpected since the tumors were selected for unambiguous morphological features concordant with clinical behavior. Importantly, IPI score was not found to be a very strong discriminative factor for clinical behavior.

The FL-stratification profile (FIG. 2b) contains genes significantly up-regulated in the aggressive phase of the disease that are involved in cell cycle control (e.g. CCNE2, CCNA2, CDK2, CHEK1, MCM7) and DNA-synthesis (e.g. TOP2A, POLD3A, HMGA1, POLE2, GMPS, CTPS) as well as genes reflecting increased metabolism (FRSB, RARS, HK2, LDHA). Genes involved in signal transduction reflecting activation of several signaling pathways are differentially expressed (e.g. FRZB, HCFCR1, PIK4CA, MAPK1). Genes derived from the reactive infiltrate of T-cells and macrophages (CD3D, CXCL12, TM4SF2) were up-regulated in the indolent phase of the disease as expected from immunohistochemical data from corresponding paraffin-embedded material of all samples, showing 40-70% T-cell infiltration in FL and 20-50% T-cell infiltration in DLBCL.

Validation of the FL-Stratification Profile in an Independent Series of Transformed FL

To validate the classifier, an additional independent set of 58 FL samples was investigated: 40 morphological/clinical indolent samples and 18 samples from the morphologically/clinically aggressive phase of the disease. Like the training series, cases were selected for concordant histological grade and clinical behavior. The gene-expression ratios of the 81 genes for these malignancies and their correlation coefficients of the malignancy samples with the average expression of these 81 genes with either “indolent” or “aggressive” samples of the training series are shown in FIG. 3. The 18 samples with values above the threshold were assigned to the high-grade category; the 40 samples below the threshold were assigned to the low-grade category. When compared to morphological and clinical data (FIG. 3b), this resulted in 4 incorrect out of 58 classifications: two false negatives, i.e. morphologically/clinically aggressive samples classified as molecular low-grade and 2 false positives, i.e. morphologically/clinically indolent classified as molecular high-grade (93% accuracy).

Performance of the FL-Stratification Profile in Histologically Difficult Cases.

To further confirm the performance of the FL-stratification classifier, 18 diagnostically difficult cases with ambiguous morphological features were selected that precluded a meaningful decision (based on grade) by the pathologist; clinical behavior was determined in retrospect as in the training and validation series. The actual clinical course as predicted by the FL-stratification classifier revealed 2 samples classified as high-grade and 16 samples as low-grade. One of these samples was incorrectly classified; this patient was clinically assigned as having aggressive disease whereas the FL-stratification classifier predicted low-grade disease (FIG. 4, Panel A). Of these 18 patients IPI scores at the time of biopsy indicated low/low-intermediate risk in 10 patients (FIG. 4a, right panel white bars) and high/high-intermediate risk in 4 patients (black bars); in 4 patients no scores could be assigned due to lack of data (gray bars).

To evaluate the notion that the 81-gene profile reflects the actual clinical behavior at the time of biopsy rather than predicts future transformation, an additional set of six patients was selected who had morphologically evident indolent, grade 1 disease in their biopsy samples and had indolent disease course at the time of the biopsy, but who presented with full-blown morphologically and clinically aggressive disease within a period of 10 months. The classifier marked 5 of 6 cases as low-grade disease in support of this hypothesis.

We have developed a FL-stratification gene expression profile to assess the actual clinical behavior in follicular lymphoma to replace current insufficient morphological and clinical methods. The classifier of 81 genes was developed in a paired-samples series of 12 FL patients who later transformed to aggressive disease to discriminate at 100% accuracy. In an independent validation series of 40 patients in a morphological/clinical indolent disease phase and 18 patients with a morphological/clinical aggressive disease phase, the molecular profile showed an accuracy of 93%. Only samples with unequivocal morphological features of indolent FL (FL grade 1 or 2) and aggressive disease (FL grade 3B or DLBCL morphology) were included, to select for an expression profile that is optimized for genes that are involved in clinically relevant transformation to the aggressive phase of the disease. The profile is therefore most relevant in the stratification of FL. This profile applies to FL and transformed FL including DLBCL, but excludes de novo DLBCL. It should be noted that de novo DLBCL represents a different disease.

The selection of the morphological spectrum of samples used in the training and the validation set (FL grade 1 and 2, and FL grade 3B and DLBCL) has a good reproducibility among hematopathologists and accounts for the significant correlation of morphological stratification and clinical course in large series. However, in practice, the largest problem is in “borderline” cases that show heterogeneity with respect to the number of large transformed cells or that contain blast-like cells with a variant morphology. In these patients, who represent approximately 10-30% of all FL biopsies, the pathologist cannot provide meaningful information to base treatment strategies on. In view of the frequent relapsing nature of the disease, histological grading problems are encountered in the far majority of the patients. We show that the genes selected in a profile that is based on the extremes of the spectrum of FL, is also of predictive relevance in the morphologically “gray zone”. Our molecular profile proves it's value mostly in this group of FL, as was supported by the findings in a test series containing this type of ambiguous cases, in which our FL-stratification profile adequately assessed the clinical behavior at the disease episode at the moment of biopsy in 17/18 patients. Notably, of the 16 patients that were classified as molecular low-grade, 5 of these patients were over-treated with aggressive therapy if currently used methods to guide therapy are compared to the stratification profile (data not shown). IPI scores in these patients showed mainly low and low-intermediate risk scores that would not add significantly as a guide in the choice of therapy. Our gene-expression-based profile outperforms IPI classification in all cases. Especially in difficult cases, the profile outperforms histological grading and significantly adds to the meaningful stratification of FL patients, and prevents patients from unnecessary aggressive treatment.

Functional annotation for the 81 genes in the FL-stratification classifier and in the full set of genes that are differentially expressed in indolent and aggressive disease provides more insight into the biological mechanisms underlying transformation in FL. Genes involved in cell cycle control, DNA synthesis and metabolism are significantly up-regulated in the aggressive phase of the disease as expected. The higher density of the T-cell infiltrate in low-grade FL as compared to high-grade disease is reflected by several T-cell related genes (CD3, CD2, CD69). However, genes related to T-cell and macrophage activation including several chemokine receptors (CCR1, CCL3, CCL5, CCL8, AKAP12, ILF3, GEM) are significantly upregulated upon transformation, indicating an important biological role. Notably, specific antagonists to several of the above mentioned chemokine receptors are available and offer attractive therapeutic interventions as do other individual gene products that are differentially expressed in the aggressive phase for which specific antagonistic and agonistic compounds have been developed (e.g. to the negative regulatory LPA-receptor Edg-2).

Our data show that on the basis of our FL-stratification profile, we are now capable of more reliably stratifying FL for low-grade and high-grade disease than by morphological grading and clinical prognostic parameters. The FL-stratification profile provides an important improvement and is an essential aid to guide the choice of therapy in FL patients to provide optimal disease control, optimal failure-free survival and quality-of-life.

TABLE 1
Criteria for retrospective assessment of the actual clinical behavior of
at any stage of the disease in follicular lymphoma patients.

| Indolent clinical behavior | Aggressive clinical behavior |
|---|---|
| Slowly progressive or waxing and | Rapid progression/enlargement of a |
| waning disease during watchful | tumor mass at one or more |
| waiting during minimally 3 months | localizations within 3 months |
| OR | AND/OR |
| Upon treatment: good tumor response | Development of B-symptoms and |
| (CR*/PR†)³⁶) after treatment with | LDH elevation after previously |
| CVP‡ or chlorambucil or | normal levels in the preceding |
| chlorambucil/prednisone or | 3 months |
| fludarabin in first line or second line. | AND/OR |
| AND | Progressive disease during |
| Normal levels of LDH§ and no B- | successive treatment with two or |
| symptoms^{\|\|} | more of the following treatment |
| | protocols CVP, chlorambucil, |
| | chlorambucil/prednisone and |
| | fludarabin. |

*CR: Complete remission;
†PR: partial remission;
‡CVP: cyclophosphomide, vincristine, prednisone;
§LDH: lactate dehydrogenase;
^{||}B-symptoms: more than 10% weight loss within 6 months, night sweats, fever more than 38° C.

TABLE 2

| | | | |
|---|---|---|---|
| NKI_ID | genbank | Gene Symbol | Description |

| | | | | |
|---|---|---|---|---|
| 19956 | AA465180 | FRSB | phenylalanyl-tRNA synthetase | |
| | | | beta-subunit | |
| 32004 | AI688220 | MCM7 | minichromosome maintenance | |
| | | | deficient (S. cerevisiae) 7 | |
| 5076 | W80964 | Ests | ESTs, Weakly similar to | GGTATTTTACATTTTATTTTGTTTTTTAGACAAATGTTC |
| | | | KIAA1353 protein [H. sapiens] | ATTGTGACTCACAGAATTGATTTCATGCCTCACTAATGG |
| | | | | GTCTCACATATACCATGAAAAATACTGATATAGAGACAT |
| | | | | TTTGTGCCTTTGCTGTGTTGGTACCTTGAGTATGTCTGC |
| | | | | TTGATGATGAAAGAGACTGGCTGATCTGCTAGACTGTTG |
| | | | | ACTTGTATAAGAACATCTTGAAGTGGTGGGAATTTATCT |
| | | | | TAGATTTTCATTCCTGGATCATTTTTATGGTCTGATCCA |
| | | | | GTGAGGAGCCAATGAGGTTTTCCATGGCTAGGTCAAATA |
| | | | | TCAGGTTGGAGTTGATGGGATATTTTTATAGAACTTTCC |
| | | | | CCGGAATGCAACCCTTGACCTCCAGCCCCAGCCGCAGCG |
| | | | | AANCCGGGCCGGGGNCCCANCGCTGCAAGCCT |
| 9222 | AA600217 | ATF4 | activating transcription factor 4 | |
| | | | (tax-responsive enhancer | |
| | | | element B67) | |
| 17262 | AA706968 | ZWINT | ZW10 interactor | |
| 7650 | W52339/ | Ests | Soares_senescent_fibroblasts_ | CACAAAATAAAAATCACTAAAAATTCCCACAAATCTTGT |
| | AA284243 | | NbHSF Homo sapiens cDNA | TTCTGGCACTTTAGAAAAACTGCAAAAAAATACGTAATA |
| | | | clone IMAGE 325583 | AAGAATACATATATATATATCTACACACAAATTATATAT |
| | | | | CTATCTATCTATACAGCGGAACCACAAGAGAGACTGAGG |
| | | | | AAGGCCTGGAGCAGGGGCAGAGGTGACGACAGTGCCCCT |
| | | | | ATTCCTTAACCCATACTCCTCTGAGGCAAACAGGCATGG |
| | | | | GAAAATGGAAGGGTTGAGGATGGACCGGAGAATTGGAAC |
| | | | | TTCAGAATAGGTCAAAATTCCAAAACCATGGACATTTTT |
| | | | | TTTTGGGAGAATTGAGAUGTAGACATTTTTTTTTTCTTA |
| | | | | AATATGATCAAGGAAAATAGCTTCCAGAATGTGGTGGTT |
| | | | | CTGGGCAACAAATGAGATTGTGGCGACGTGGAGATTAAA |
| | | | | ATATATGTATTTGAGCTGGGGAATTTGAATATTGTGAGT |
| | | | | TTCAGATGTTGGAAATTTGGGATTTTGCAGT |
| 31656 | AI203233 | AND-1 | AND-1 protein | |
| 5088 | N64781 | PRO1659 | PRO1659 protein | |
| 2940 | AA504348 | TOP2A | topoisomerase (DNA) II alpha | |
| | | | (170 kD) | |
| 8214 | AA055946 | CD3D | CD3D antigen, delta | |
| | | | polypeptide (TiT3 complex) | |
| 8574 | W68396 | KIAA0096 | KIAA0096 protein | |
| 2334 | AA608568 | CCNA2 | cyclin A2 | |
| 17328 | AA775257 | ITM2A | integral membrane protein 2A | |
| 12690 | AA620485 | LOC51203 | clone HQ0310 PRO0310p1 | |
| 1416 | AA447115 | SDF1 | stromal cell-derived factor 1 | |
| 23717 | H23211 | MGC3077 | hypothetical protein MGC3077 | |
| 8315 | AA421269 | PIK4CA | phosphatidylinositol 4-kinase, | |
| | | | catalytic, alpha polypeptide | |
| 15197 | AA478596 | KIAA0854 | KIAA0854 protein | |
| 27773 | R21530 | MCM4 | minichromosome maintenance | |
| | | | deficient (S. cerevisiae) 4 | |
| 21113 | AI005515 | HK2 | hexokinase 2 | |
| 20693 | AA206225 | ESTs | Stratagene neuroepithelium | CAGTGAGAAAAATAAAGTTTATTTCTTGTACAGAAGTAA |
| | | | (#937231) Homo sapiens | TTCCAATTCAGGTTTTAAGTCTGAACCTTCAAACTTGGG |
| | | | cDNA clone IMAGE 645662 | AAGTTTTTTTCCAAGTCATCAGCAAGCGCCTTATCAACT |
| | | | | TCACGTAGGATTTTAATAAAATCTTCCACCACAGGGCTA |
| | | | | TAGAAAGGAAGAAGGTTTTGAAATGCAGAGGAACACTTC |
| | | | | TGTTTGCTCAACTGGCCTTCCTTTACTCCCTGAGCCACC |
| | | | | GTCTCCTTTTTCTATCTTTAGATACTCTTGCTCAGTCTG |
| | | | | TGAGGCATAGATGGCAGACAACACTGAAAAAACAGAGGC |
| | | | | TAGCACTGTTTTCTGTTCTTGAAGAATGATGGGTGGATC |
| | | | | ATCAGACTGGCAGAATTCATGGCAGACCAGGTCAAAAAG |
| | | | | TAAATTCCAAATGTCTTTTCCAGTGTCGGACAATGTACA |
| | | | | ATTGCTGAATTCCTCATCCACTGTAGTAAGCAGTTGTAA |
| | | | | AATGTGCTGTAAACATCAAGCCATTCTGGATTTTCAGAA |
| | | | | CGTACTTGTTTGGCAGCTCAAGTATACAGGGCACAAGCC |
| | | | | GAAACACTGGCTGCTCTCAGACTCTTCCGGGGTTGGTCC |
| | | | | AGAGGCTGAGCAGCCCATTCTGACCCATCTAACATAGTT |
| | | | | CTCACCAATCAAGGCTGTCACACTCCCACTTNACAGCAG |
| | | | | CACATGTGACTGCTATGAGCAA |
| 827 | R83876 | ABCB10 | ATP-binding cassette, sub- | |
| | | | family B (MDR/TAP), member | |
| | | | 10 | |
| 1541 | T65786 | SFRS1 | splicing factor, arginine/serine- | |
| | | | rich 1 (splicing factor 2, | |
| | | | alternate splicing factor) | |
| 8747 | T73558 | DNASE1L3 | deoxyribonuclease I-like 3 | |
| 11567 | AA428607 | TSPYL | TSPY-like | |
| 8615 | AA629558 | SLBP | stem-loop (histone) binding | |
| | | | protein | |
| 1559 | R43817 | NPY1R | neuropeptide Y receptor Y1 | |
| 20015 | AA520999 | CCNE2 | cyclin E2 | |
| 2135 | AA448261 | HMGIY | high-mobility group | |
| | | | (nonhistone chromosomal) | |
| | | | protein isoforms I and Y | |
| 2602 | W35283 | DC49 | Homo sapiens DC49 mRNA, | |
| | | | complete cds | |
| 18658 | AA485732/ | Ests | Soares ovary tumor NbHOT | ACTGTTCGTTCATTTATTCATTCAACAAACATTATATGA |
| | AI732826 | | Homo sapiens cDNA clone | GCACCCACTATGTGCCAGGCACAGTGTTAGGTCCCACAC |
| | | | IMAGE | ATACAGAGCTGAATCAGGCATAGTCCTTACTTCAGGAAT |
| | | | | TTCACAGTCTAGTAGTAGATACAGACATTTTAAAAGGTA |
| | | | | ATTATAAAATAAAATGGTAATTATGGTACAATACGGTAA |
| | | | | ATGAAATAATAGAGATATACCTTGGGCATTAAGGAACCA |
| | | | | CAATAGAGAGGCATCTACCCAAGCCTAGAATATAGGACA |
| | | | | GGTTTGCTGGAAGAGCTGGGTTCTGGGCTGAATAAACAG |
| | | | | CATGATATAAGCTGAGAACTAAAGGTAATTTTGTCCAGA |
| | | | | GACAGGGAAAGGAGGAGTTAAGGCATCAGATGTAGGCAG |
| | | | | GAGGGAGGTCACAGTGGAATTTGAATTAATCTGTGGCAC |
| | | | | AGCATTTCTGCCTGGGGTCAAGTTGGAGGTCTGCAGTTA |
| | | | | TAAATTCTTGGATATACAAGTTTTCTG |
| 7354 | W93379 | NEK2 | NIMA (never in mitosis gene | |
| | | | a)-related kinase 2 | |
| 3220 | T66935 | DKFZp762E1312 | hypothetical protein | |
| | | | DKFZp762E1312 | |
| 18130 | H19826 | HPCAL1 | hippocalcin-like 1 | |
| 16882 | AA894927 | ASNS | asparagine synthetase | |
| 712 | AA041400 | CDC4L | cell division cycle 4-like | |
| 7444 | R26046 | ILF3 | interleukin enhancer binding | |
| | | | factor 3, 90 kD | |
| 16906 | AA894577 | NOP56 | nucleolar protein (KKE/D | |
| | | | repeat) | |
| 628 | N59764 | GMPS | guanine monphosphate | |
| | | | synthetase | |
| 31918 | AI653017 | CDK2 | cyclin-dependent kinase 2 | |
| 1384 | AA504348 | TOP2A | topoisomerase (DNA) II alpha | |
| | | | (170 kD) | |
| 2259 | AA453471 | K-ALPHA-1 | tubulin, alpha, ubiquitous | |
| 17445 | AA291773 | TETRAN | tetracycline transporter-like | |
| | | | protein | |
| 3189 | H80103 | HSPC154 | HSPC154 protein | |
| 2961 | R63543 | DXS6984E | p75NTR-associated cell death | |
| | | | executor; ovarian granulosa | |
| | | | cell protein (13 kD) | |
| 31539 | AI263201 | SDF1 | stromal cell-derived factor 1 | |
| 8169 | AA485380 | SMCX | SMC (mouse) homolog, X | |
| | | | chromosome | |
| 11025 | W60983 | KIAA0669 | KIAA0669 gene product | |
| 8361 | N33274 | ADE2H1 | multifunctional polypeptide | |
| | | | similar to SAICAR synthetase | |
| | | | and AIR carboxylase | |
| 8769 | AA669637 | B4-2 | proline-rich protein with | |
| | | | nuclear targeting signal | |
| 8793 | N90783 | P2Y5 | purinergic receptor (family A | |
| | | | group 5) | |
| 5511 | AA026682 | TOP2A | topoisomerase (DNA) II alpha | |
| | | | (170 kD) | |
| 28359 | AI141490 | PTX3 | pentaxin-related gene, rapidly | |
| | | | induced by IL-1 beta | |
| 27879 | AA454111 | FRZB | frizzled-related protein | |
| 8607 | AA478066 | PKMYT1 | membrane-associated | |
| | | | tyrosine- and threonine-specific | |
| | | | cdc2-inhibitory kinase | |
| 9213 | N93505 | TM4SF2 | transmembrane 4 superfamily | |
| | | | member 2 | |
| 8648 | AA074535 | HPIP | hematopoietic PBX-interacting | |
| | | | protein | |
| 908 | N53057 | CHEK1 | CHK1 (checkpoint, S. pombe) | |
| | | | homolog | |
| 1724 | AA449762 | ILF3 | interleukin enhancer binding | |
| | | | factor 3, 90 kD | |
| 15254 | AA447696 | LOC51646 | CGI-127 protein | |
| 1868 | AA504204 | POLD3 | polymerase (DNA directed), | |
| | | | delta 3 | |
| 9014 | AA176957 | NEB | nebulin | |
| 8756 | AA701455 | CENPF | centromere protein F | |
| | | | (350/400 kD, mitosin) | |
| 590 | AA477501 | KIAA0042 | KIAA0042 gene product | |
| 5809 | AA131909 | FLJ10540 | hypothetical protein FLJ10540 | |
| 5833 | W68585 | HIS1 | HMBA-inducible | |
| 26185 | AA936181 | DKFZp762E1312 | hypothetical protein | |
| | | | DKFZp762E1312 | |
| 17425 | AA521015 | DNAJC4 | DnaJ (Hsp40) homolog, | |
| | | | subfamily C, member 4 | |
| 17017 | AA777192 | LAMR1 | laminin receptor 1 (67 kD, | |
| | | | ribosomal protein SA) | |
| 1873 | AA454572 | Ests | Soares ovary tumor NbHOT | ATATTTTATTCAACTTTATTAAAAATTAAAACTACAGAA |
| | | | Homo sapiens cDNA clone | ACCAAACCGAATGCACAAATAAGAGAGTGGGGAAACAGA |
| | | | IMAGE 809530 | CAGGGAGCAATGGCAGCTGAT |
| 2107 | T59334 | CSRP2 | cysteine and glycine-rich | |
| | | | protein 2 | |
| 2467 | AA449667 | MFGE8 | milk fat globule-EGF factor 8 | |
| | | | protein | |
| 5503 | AA011414 | FGA | fibrinogen, A alpha polypeptide | |
| 31159 | W44416 | CTPS | CTP synthase | |
| 1609 | AA497029 | LDHA | lactate dehydrogenase A | |
| 28177 | AA774740 | RARS | arginyl-tRNA synthetase | |
| 6229 | AA464180 | HBEX2 | uncharacterized hypothalamus | |
| | | | protein HBEX2 | |
| 15157 | AA448664 | POLE2 | polymerase (DNA directed), | |
| | | | epsilon 2 | |
| 9013 | AA676598 | IFRD1 | interferon-related | |
| | | | developmental regulator 1 | |
| 20365 | AA278865 | ESTs | NCI_CGAP_GCB1 Homo | TTTTTTCTTCTCAAATTTTTAATGGTTCTCCACGGTATG |
| | | | sapiens cDNA clone IMAGE | CTAGGTCCTTATTTTCTCATTGGCTGTAATTTGACAAGT |
| | | | 703559 | TCTCCAAAAGCACTATCATGAACTTAATTAGATAATACT |
| | | | | TTCCAGCTGTACTTAAATCAGAAGGATCGTAGGCCTGAT |
| | | | | AAGCAGGTCAACCTGAAGCTACCAGGCAGCTCTGGAGGC |
| | | | | TCCCGTGGCCATAGCTAACAGTGCACAGGAGGGCCCCTG |
| | | | | GCCAGGATGAGCCCTCAGAACCTTTTAGAGAGAGTGAAA |
| | | | | TTCAGGGTCTACTGGGTGTCCAAGCTGCAGAGTGGAGCG |
| | | | | TGAGAGGTCAGCTGCCATGTGATAGAAAGAGCTGACATA |
| | | | | AGAGAAAAGACCCCCACACAGTGAAAAGCTGGAAGAGAA |
| | | | | ACAAAACTCACAACCTGACAGCAGAGTTCCTGGUCCAAC |
| | | | | TGTTGAGACCTGTCTCTACCTCCAATGGTCACAGTATAT |
| | | | | AGTTACCCAGGCCTG |
| 8155 | AA043501 | MAF | v-maf musculoaponeurotic | |
| | | | fibrosarcoma (avian) oncogene | |
| | | | homolog | |

TABLE 3

Genbank

| | |
|---|---|
| Classifier | Accession |

| | | | | | |
|---|---|---|---|---|---|
| Order | NKI Id | No. | Symbol | Source Description | Sequence |

| | | | | | |
|---|---|---|---|---|---|
| 1 | 11567 | AA428607 | TSPYL1 | TSPY-like 1 | |
| 2 | 8648 | AA074535 | PBXIP1 | Pre-B-cell leukemia transcription | |
| | | | | factor interacting protein 1 | |
| 3 | 18658 | AA485732\| | ESTs | Soares ovary tumor NbHOT Homo | ACTGTTCGTTCATTTATTCATTCA |
| | | AI732826 | | sapiens cDNA clone IMAGE | ACAAACATTATATGAGCACCCACT |
| | | | | | ATGTGCCAGGCACAGTGTTAGGTC |
| | | | | | CCACACATACAGAGCTGAATCAGG |
| | | | | | CATAGTCCTTACTTCAGGAATTTC |
| | | | | | ACAGTCTAGTAGTAGATACAGACA |
| | | | | | TTTTAAAAGGTAATTATAAAATAA |
| | | | | | AATGGTAATTATGGTACAATACGG |
| | | | | | TAAATGAAATAATAGAGATATACC |
| | | | | | TTGGGCATTAAGGAACCACAATAG |
| | | | | | AGAGGCATCTACCCAAGCCTAGAA |
| | | | | | TATAGGACAGGTTTGCTGGAAGAG |
| | | | | | CTGGGTTCTGGGCTGAATAAACAG |
| | | | | | CATGATATAAGCTGAGAACTAAAG |
| | | | | | GTAATTTTGTCCAGAGACAGGGAA |
| | | | | | AGGAGGAGTTAAGGCATCAGATGT |
| | | | | | AGGCAGGAGGGAGGTCACAGTGGA |
| | | | | | ATTTGAATTAATCTGTGGCACAGC |
| | | | | | ATTTCTGCCTGGGGTCAAGTTGGA |
| | | | | | GGTCTGCAGTTATAAATTCTTGGA |
| | | | | | TATACAAGTTTTCTG |
| 4 | 15157 | AA448664 | POLE2 | Polymerase (DNA directed), | |
| | | | | epsilon 2 (p59 subunit) | |
| 5 | 2961 | R63543 | NGFRAP1 | Nerve growth factor receptor | |
| | | | | (TNFRSF16) associated protein 1 | |
| 6 | 18130 | H19826 | HPCAL1 | Hippocalcin-like 1 | |
| 7 | 31656 | AI203233 | WDHD1 | WD repeat and HMG-box DNA | |
| | | | | binding protein 1 | |
| 8 | 15197 | AA478596 | ZHX2 | Zinc fingers and homeoboxes 2 | |
| 9 | 9014 | AA176957 | NEB | Nebulin | |
| 10 | 1868 | AA504204 | POLD3 | Polymerase (DNA-directed), delta | |
| | | | | 3, accessory subunit | |
| 11 | 28359 | AI141490 | PTX3 | Pentraxin-related gene, rapidly | |
| | | | | induced by IL-1 beta | |
| 12 | 9013 | AA676598 | IFRD1 | Interferon-related developmental | |
| | | | | regulator 1 | |
| 13 | 1559 | R43817 | NPY1R | Neuropeptide Y receptor Y1 | |
| 14 | 7650 | W52339\| | ZBTB4 | Zinc finger and BTB domain | |
| | | AA284243 | | containing 4 | |
| 15 | 8361 | N33274 | PAICS | Phosphoribosylaminoimidazole | |
| | | | | carboxylase, | |
| | | | | phosphoribosylaminoimidazole | |
| | | | | succinocarboxamide synthetase | |
| 16 | 5076 | W80964 | LOC285550 | Hypothetical protein LOC285550 | |
| 17 | 3189 | H80103 | C18orf55 | Chromosome 18 open reading | |
| | | | | frame 55 | |
| 18 | 908 | N53057 | CHEK1 | CHK1 checkpoint homolog | |
| | | | | (S. pombe) | |
| 19 | 5503 | AA011414 | FGA | Fibrinogen, A alpha polypeptide | |
| 20 | 8169 | AA485380 | SMCX | SMC (mouse) homolog, X | |
| | | | | chromosome | |
| 21 | 23717 | H23211 | C7orf24 | Chromosome 7 open reading | |
| | | | | frame 24 | |
| 22 | 8574 | W68396 | SNRK | SNF related kinase | |
| 23 | 27879 | AA454111 | FRZB | Frizzled-related protein | |
| 24 | 2467 | AA449667 | MFGE8 | Milk fat globule-EGF factor 8 | |
| | | | | protein | |
| 25 | 1384 | AA504348 | TOP2A | Topoisomerase (DNA) II alpha | |
| | | | | 170 kDa | |
| 26 | 17328 | AA775257 | ITM2A | Integral membrane protein 2A | |
| 27 | 8747 | T73558 | DNASE1L3 | Deoxyribonuclease I-like 3 | |
| 28 | 9213 | N93505 | TM4SF2 | Tetraspanin 7 | |
| 29 | 8214 | AA055946 | CD3D | CD3D antigen, delta polypeptide | |
| | | | | (TiT3 complex) | |
| 30 | 12690 | AA620485 | NUSAP1 | Nucleolar and spindle associated | |
| | | | | protein 1 | |
| 31 | 2259 | AA453471 | GM2A | GM2 ganglioside activator | |
| 32 | 5833 | W68585 | HIS1 | HMBA-inducible | |
| 33 | 8615 | AA629558 | SLBP | Stem-loop (histone) binding | |
| | | | | protein | |
| 34 | 8756 | AA701455 | CENPF | Centromere protein F, 350/400 ka | |
| | | | | (mitosin) | |
| 35 | 26185 | AA936181 | DKFZp762E1312 | Hypothetical protein | |
| | | | | DKFZp762E1312 | |
| 36 | 31159 | W44416 | CTPS | CTP synthase | |
| 37 | 7354 | W93379 | NEK2 | NIMA (never in mitosis gene a)- | |
| | | | | related kinase 2 | |
| 38 | 628 | N59764 | GMPS | guanine monphosphate | |
| | | | | synthetase | |
| 39 | 8155 | AA043501 | MAF | V-maf musculoaponeurotic | |
| | | | | fibrosarcoma oncogene homolog | |
| | | | | (avian) | |
| 40 | 5068 | N64781 | SLC43A3 | Solute carrier family 43, member 3 | |
| 41 | 8607 | AA478066 | PKMYT1 | Protein kinase, membrane | |
| | | | | associated tyrosine/threonine 1 | |
| 42 | 17425 | AA521015 | DNAJC4 | DnaJ (Hsp40) homolog, subfamily | |
| | | | | C, member 4 | |
| 43 | 2334 | AA608568 | CCNA2 | Cyclin A2 | |
| 44 | 16906 | AA894577 | NOL5A | Nucleolar protein 5A (56 kDa with | |
| | | | | KKE/D repeat) | |
| 45 | 19956 | AA465180 | FARSLB | Phenylalanine-tRNA synthetase- | |
| | | | | like, beta subunit | |
| 46 | 2135 | AA448261 | HMGA1 | High mobility group AT-hook 1 | |
| 47 | 1724 | AA449762 | ILF3 | Interleukin enhancer binding factor | |
| | | | | 3, 90 kDa | |
| 48 | 15254 | AA447696 | YPEL5 | Yippee-like 5 (Drosophila) | |
| 49 | 21113 | AI005515 | HK2 | Hexokinase 2 | |
| 50 | 16882 | AA894927 | ASNS | Asparagine synthetase | |
| 51 | 31539 | AI263201 | CXCL12 | Chemokine (C-X-C motif) ligand | |
| | | | | 12 (stromal cell-derived factor 1) | |
| 52 | 8315 | AA421269 | PIK4CA | Phosphatidylinositol 4-kinase, | |
| | | | | catalytic, alpha polypeptide | |
| 53 | 712 | AA041400 | LRBA | LPS-responsive vesicle trafficking, | |
| | | | | beach and anchor containing | |
| 54 | 9222 | AA600217 | ATF4 | Activating transcription factor 4 | |
| | | | | (tax-responsive enhancer element | |
| | | | | B67) | |
| 55 | 1416 | AA447115 | CXCL12 | Chemokine (C-X-C motif) ligand | |
| | | | | 12 (stromal cell-derived factor 1) | |
| 56 | 31918 | AI653017 | CDK2 | Cyclin-dependent kinase 2 | |
| 57 | 27773 | R21530 | MCM4 | minichromosome maintenance | |
| | | | | deficient (S. cerevisiae) 4 | |
| 58 | 1541 | T65786 | SFRS1 | Splicing factor, arginine/serine-rich | |
| | | | | 1 (splicing factor 2, alternate | |
| | | | | splicing factor) | |
| 59 | 20693 | AA206225 | LOC113174 | Hypothetical protein BC012010 | |
| 60 | 6229 | AA464180 | BEX2 | Brain expressed X-linked 2 | |
| 61 | 8769 | AA669637 | PNRC1 | Proline-rich nuclear receptor | |
| | | | | coactivator 1 | |
| 62 | 8793 | N90783 | P2RY5 | Purinergic receptor P2Y, G-protein | |
| | | | | coupled, 5 | |
| 63 | 827 | R83876 | ABCB10 | ATP-binding cassette, sub-family | |
| | | | | B (MDR/TAP), member 10 | |
| 64 | 1873 | AA454572 | ESTs | Soares ovary tumor NbHOT Homo | ATATTTTATTCAACTTTATTAAAA |
| | | | | sapiens cDNA clone IMAGE | ATTAAAACTACAGAAACCAAACCG |
| | | | | 809530 | AATGCACAAATAAGAGAGTGGGGA |
| | | | | | AACAGACAGGGAGCAATGGCAGCT |
| | | | | | GAT |
| 65 | 3220 | T66935 | DKFZp762E1312 | Hypothetical protein | |
| | | | | DKFZp762E1312 | |
| 66 | 2940 | AA504348 | TOP2A | Topoisomerase (DNA) II alpha | |
| | | | | 170 kDa | |
| 67 | 2602 | W35283 | LAT1-3TM | LAT1-3TM protein | |
| 68 | 1609 | AA497029 | LDHA | Lactate dehydrogenase A | |
| 69 | 5809 | AA131909 | C10orf3 | Chromosome 10 open reading | |
| | | | | frame 3 | |
| 70 | 28177 | AA774740 | RARS | Arginyl-tRNA synthetase | |
| 71 | 5511 | AA026682 | TOP2A | topoisomerase (DNA) II alpha | |
| | | | | (170 kD) | |
| 72 | 17262 | AA706968 | ZWINT | ZW10 interactor | |
| 73 | 32004 | AI688220 | MCM7 | MCM7 minichromosome | |
| | | | | maintenance deficient 7 | |
| | | | | (S. cerevisiae) | |
| 74 | 2107 | T59334 | CSRP2 | Cysteine and glycine-rich protein 2 | |
| 75 | 20365 | AA278865 | C13orf3 | Chromosome 13 open reading | |
| | | | | frame 3 | |
| 76 | 11025 | W60983 | KIAA0669 | TSC22 domain family 2 | |
| 77 | 7444 | R26046 | ILF3 | Interleukin enhancer binding factor | |
| | | | | 3, 90 kDa | |
| 78 | 590 | AA477501 | KIF14 | Kinesin family member 14 | |
| 79 | 20015 | AA520999 | CCNE2 | Cyclin E2 | |
| 80 | 17017 | AA777192 | POLR2I | Polymerase (RNA) II (DNA | |
| | | | | directed) polypeptide I, 14.5 kDa | |
| 81 | 17445 | AA291773 | TETRAN | Tetracycline transporter-like | |
| | | | | protein | |

1. A method for detecting and/or staging a malignant functional cell, said method comprising:
measuring an expression profile of said malignant functional cell and a non-malignant cell, wherein said non-malignant cell and said malignant functional cell are capable of interacting.

2. The method according to claim 1, further comprising:
determining the presence and/or amount of expression product of at least two reporter genes in a sample comprising said malignant functional cell and at least one non-malignant cell.

3. The method according to claim 2, wherein at least one reporter gene is involved with a proliferation/apoptosis pathway, a metabolism pathway, an immune competence pathway and/or a T cell infiltration pathway.

4. The method according to claim 2, wherein said expression product comprises mRNA.

5. The method according to claim 2, wherein said expression product comprises protein.

6. The method according to claim 1, further comprising determining in a sample comprising said non-malignant cell, the presence and/or amount of an expression product of at least 5 reporter genes listed in Table 2.

7. The method according to claim 6, comprising determining in a sample comprising said non-malignant cell the presence and/or amount of expression product of at least 10 reporter genes listed in Table 2.

8. The method according to claim 7, comprising determining in a sample comprising said non-malignant cell the presence and/or amount of expression product of at least 30 reporter genes listed in Table 2.

9. The method according to claim 8, comprising determining in a sample comprising said non-malignant cell the presence and/or amount of expression product of at least 50 reporter genes listed in Table 2.

10. The method according to claim 9, comprising determining in a sample comprising said non-malignant cell the presence and/or amount of expression product of each of the reporter genes listed in Table 2.

11. The method according to claim 1, wherein said malignant functional cell is capable of producing an antibody, enzyme and/or hormone.

12. The method according to claim 1, wherein said malignant functional cell comprises a B cell.

13. The method according to claim 1, wherein said malignant functional cell comprises a T cell.

14. The method according to claim 1, wherein said malignant functional cell comprises a follicular lymphoma cell.

15. The method according to claim 1, wherein said malignant functional cell comprises a mucosa-associated lymphoid tissue lymphoma cell.

16. The method according to any claim 1, wherein said malignant functional cell comprises a cell of a Grawitz tumour.

17. The method according to claim 1, wherein said malignant functional cell comprises a prostate cancer cell.

18. The method according to claim 1, wherein said non-malignant cell comprises a T cell.

19. The method according to claim 1, wherein said non-malignant cell comprises a T helper cell.

20. An array comprising nucleic acid molecules capable of specifically binding to at least 5 reporter genes listed in Table 2.

21. The array of claim 20, comprising nucleic acid molecules capable of specifically binding to at least 10 reporter genes listed in Table 2.

22. The array of claim 21, comprising nucleic acid molecules capable of specifically binding to at least 30 reporter genes listed in Table 2.

23. The array of claim 22, comprising nucleic acid molecules capable of specifically binding to at least 50 reporter genes listed in Table 2.

24. The array of claim 23, comprising nucleic acid molecules capable of specifically binding to each of the reporter genes listed in Table 2.

25. A microarray comprising the array of claim 20.

26. The array according to claim 20, comprising at least one probe specific for a nucleic acid molecule of rows or classifier order 1-5 of Table 3.

27. The array according to claim 20, comprising at least one probe specific for a nucleic acid molecule of rows or classifier order 6-10 of Table 3.

28. The array according to claim 26, comprising probes specific for the nucleic acid molecules of rows or classifier order 1-5 of Table 3.

29. The array according to claim 26, comprising probes specific for the nucleic acid molecules of rows or classifier order 6-10 of Table 3.

30. The array according to claim 20, comprising nucleic acid molecules capable of specifically binding to at least 60 reporter genes listed in Table 2.

31. The array according to claim 20, further comprising a reference probe.

32. The array according to claim 31 comprising between 50 and 20,000 reference probes.

33. The array according to claim 32, comprising between 100 and 5000 reference probes.

34. A method for detecting and/or staging a malignant functional cell, said method comprising:
providing the array of claim 20 with a sample comprising expression product from said malignant functional cell and from a non-malignant cell; and
determining whether an expression profile indicative for the presence and/or stage of said malignant functional cell is obtained.

35. A method for treating a disease involving a malignant functional cell, said method comprising:
at least in part inhibiting an interaction between said malignant functional cell and a non-malignant cell.

36. The method according to claim 35, comprising at least in part inhibiting signaling of a T cell.

37. (canceled)

38. A method of treating a disease in a subject involving a malignant functional cell, said method comprising:
at least in part inhibiting signaling of a T cell in the subject so as to treat a disease involving a malignant functional cell.

39. The method of claim 38, wherein the disease involving a malignant functional cell is follicular lymphoma.

40. A method for determining whether a sample comprises a malignant functional cell, said method comprising:
providing the array of claim 20, with expression products from said sample and determining whether an expression profile of said sample on said array is indicative for the presence and/or stage of said malignant functional cell.

41. The method according to claim 40, wherein said expression products comprise RNA.
